(19) United States
(12) Patent Application Publication
(10) Pub. No.: US 2015/0165313 A1
(43) Pub. Date: Jun. 18, 2015

(54) METHOD AND SYSTEM FOR INCORPORATING PHYSIOLOGIC DATA IN A GAMING ENVIRONMENT

(71) Applicant: Proteus Digital Health, Inc., Redwood City, CA (US)

(72) Inventors: Andrew Thompson, Portola Valley, CA (US); George Savage, Portola Valley, CA (US); Erika Karplus, Silverthorne, CO (US); Lawrence Arne, Palo Alto, CA (US); Timothy Robertson, Belmont, CA (US); David O'Reilly, Palo Alto, CA (US)

(21) Appl. No.: 14/582,042

(22) Filed: Dec. 23, 2014

Related U.S. Application Data
(63) Continuation of application No. 13/124,094, filed on May 19, 2011.
(60) Provisional application No. 61/105,346, filed on Oct. 14, 2008.

(51) Int. Cl. A63F 13/212; A61B 5/00; A61B 5/0205; A61B 5/07

(52) U.S. Cl. CPC A63F 13/212; A61B 5/073; A61B 5/4833; A61B 5/02055; A61B 5/02405

(57) ABSTRACT

The present invention provides a receiving device and method for use with gaming pursuits, including, in one aspect, a personal signal receiver to communicate physiologic data, a hub to receive the physiologic data, and a gaming module to receive, directly or indirectly, the physiologic data from the hub.

### CROSS-REFERENCE TO RELATED APPLICATIONS

The present application is a continuation application of co-pending U.S. patent application Ser. No. 13/124,094, filed May 19, 2011, which is a national stage filing of PCT Application Number PCT/US09/60713, filed on Oct. 14, 2011, which claims the benefit of U.S. Provisional Patent Application 61/105,346, filed Oct. 14, 2008, the entire disclosures of which are incorporated herein by reference.

### INTRODUCTION

Gaming generally includes any of a number of structured pursuits. Such pursuits include online games in which participants access the game via software and interact over the Internet. For example, remote participants may participate in a software game in which each participant assumes the role of a fictional character or a non-fictional characters. The participants determine the actions of their characters based on their characterization, and the actions succeed or fail according to a formal system of rules and/or guidelines in the software. Within the rules, the participants can improvise freely and their choices/actions shape the direction and outcome of the game.

To illustrate, in a competitive online poker game, each participant may select a fictional poker player character. Each participant then makes choices allowed under the software rules to forward the game, e.g., decide which cards to keep, how much to bet, when to fold, etc.

To compete on a more personal level, however, personal dynamics may have to be introduced. The term “personal dynamic” is used herein to indicate one or more parameters associated with an individual, e.g., heart rate, respiration rate, etc.

To illustrate, competition and outcome in an online poker game may differ considerably if participants had information leading to detection of a change in participants' behavior or demeanor, i.e., a “tell”. The tell may give clues to a participant's assessment of his hand. Thus, the participant detecting the tell may gain an advantage if the meaning of the tell is correctly interpreted, particularly if the tell is spontaneous and reliable.

Introduction of personal dynamics into a realm of play may prove challenging. For example, the participants may be located in geographically-diverse locations from one another. The participants may not personally know each other. The participants may have no prior knowledge about one another, thus creating a vacuum of information with respect to one another.

As can be seen, techniques for observation, collection, and interpretation of data are needed to provide certain dynamics as parameters for games and other recreational events. Therefore, there is a need to obtain and utilize accurate personal data for this and other purposes.

### SUMMARY

The present invention relates generally to gaming devices as well as procurement and use of physiologic data in gaming and recreational pursuits. The present invention seeks to address at least some of the previously-discussed issues and may be broadly applicable across a variety of pursuits.

One aspect of the disclosure provides a personal signal receiver to communicate physiologic data, a hub to receive the physiologic data, and a gaming module to receive, directly or indirectly, the physiologic data from the hub.

One aspect of the disclosure provides a method which includes steps of communicating, via a personal signal receiver, physiologic data; receiving, via a hub, the physiologic data; and receiving directly or indirectly, via a physiologic gaming module, the physiologic data from the hub.

An aspect of the disclosure provides a storage medium having instructions, that when executed by a computing platform, result in execution of a method of utilizing physiologic data in conjunction with a gaming environment. The method, for example, may include steps of communicating, via a personal signal receiver, the physiologic data; receiving, via a hub, the physiologic data; and receiving directly or indirectly, via a physiologic gaming module, the physiologic data from the hub.

### BRIEF DESCRIPTION OF THE FIGURES

FIG. 1 provides an exemplary diagrammatic representation of a gaming environment.

FIG. 2 provides an exemplary diagrammatic representation of the system for incorporating physiologic data in the gaming environment of FIG. 1.

FIG. 3 illustrates an exemplary personal signal receiver of the system for incorporating physiologic data in a gaming environment of FIG. 2.

FIG. 4 illustrates exemplary physiologic data associated with the system for incorporating physiologic data in a gaming environment of FIG. 2.

FIG. 5 illustrates a hub associated with the system for incorporating physiologic data of FIG. 2.

FIG. 6 illustrates a physiologic gaming module associated with the system for incorporating physiologic data of FIG. 2.

FIG. 7 illustrates a method for incorporating physiologic data in a gaming environment.

### DETAILED DESCRIPTION

### 1.0 Overview

The method and system for incorporating physiologic data in a gaming environment provide a uniform, comprehensive framework to enable gaming pursuits having a physiologically-based component. The physiologically-based component may include, for example, physiologic data based on physiologic parameters or traits. The physiologic data may be utilized in various ways in the gaming environment. Examples include, but are not limited to, directly reporting the physiologic data in the game, using the physiologic data as a basis from which to draw an inference in a game, using the physiologic data to drive a decision factor in a game, and storing the physiologic data for future uses in a game.

The method and system for incorporating physiologic data in a gaming environment is highly scalable as standalone systems and as integratable systems. Integratable systems may be integrated, for example, with various existing systems such as device games, software games, and online games. As used herein, the term “online game” refers to any recreation or competition-based pursuit facilitated at least in part by a computer and/or gaming device and in which players are engaged via a communications network or computer network such as the Internet.

Broadly, various aspects of the method and system for incorporating physiologic data in a gaming environment include a basic complement of core components, e.g., a personal signal receiver to communicate physiologic data; a hub to receive the physiologic data; and a gaming system to receive, directly or indirectly, the physiologic data from the hub. Any one or a combination of these core components is capable of interoperation, communication, and/or integration with various components of other information/communication systems. The terms “data” and “information” are used interchangeably herein.

The personal signal receiver includes any device capable of communicating, e.g., receiving, transmitting, generating, recording, etc., physiologic data. The physiologic data include any data of a physiologic nature associated with a human or non-human being. The physiologic data include, for example, heart rate, heart rate variability, respiration rate, body temperature, temperature of local environment, three-axis measurement of activity and torso angle, as well as other physiologic data, metrics, and indicators associated with one or more individuals. The physiologic data may be communicated at various times or time intervals. For example, the communication may be real-time, i.e., in close temporal proximity to a time in which the physiologic data was generated, measured, ascertained, etc., or on an historical basis, i.e., in far temporal proximity to a time in which the physiologic data was generated, measured, ascertained, etc. In various aspects, the physiologic data may be associated with a variety of devices, e.g., cardiac device, etc.

The hub includes any hardware device, software, and/or communications component(s), as well as systems, subsystems, and combinations of the same which generally function to communicate the physiologic data. Communication of the physiologic data includes receiving, storing, manipulating, displaying, processing, and/or transmitting the physiologic data.

In various aspects, the hub also functions to communicate, e.g., receive and transmit, non-physiologic data. Example of non-physiologic data include gaming rules and data generated by a separate cardiac-related device such as an implanted pacemaker and communicated to the hub directly or indirectly, e.g., via the personal signal receiver.

Broad categories of hubs include, for example, base stations, personal communication devices, handheld devices, and mobile telephones.

For example, the hub includes a software application associated with a mobile telephone of a patient. The application and mobile telephone function to receive physiologic data from a receiver, which, in turn, receives the physiologic data directly from an individual or indirectly, e.g., via a device. Examples of devices include cardiac devices and ingestible devices. The hub stores, manipulates, and/or forwards the data, alone or in combination with other data, via a physiologic gaming module.

Embodiments of the invention include compositions having: an active agent; an identifier and a pharmaceutically acceptable carrier. In one embodiment of the present invention, an ingestible pill is made identifiable by providing an electronic microchip as part of the pill structure. In some aspects, the electronic microchip is completely encased within the pill. In this embodiment, the pill broadcasts a signal when it is dissolved in an ionic solution such as stomach fluids. The broadcasted signal is received by another device, e.g., a receiver, either inside or near the body. In turn, the receiver then records that the pill has in fact reached the stomach and is in the process of being dissolved.

Also present in the subject compositions is an identifier. The identifier may vary depending on the particular embodiment and intended application of the composition. In certain embodiments, the identifier is a component that emits a signal upon activation by a stimulus, e.g., by interrogation, upon contact with a target physiological location, etc. As such, the identifier may be an identifier that emits a signal when it contacts a target body (i.e., physiological) site. In addition or alternatively, the identifier may be an identifier that emits a signal when interrogated.

In yet other embodiments, the identifier is an inert, but identifiable marker, e.g., an engraved identifier (such as one that is fabricated from a material or materials that survive digestion). This marker may then be identified, for example, following an autopsy or forensic examination. It is possible to provide a more internal device within a pill to determine both that its surface has partially been subject to digestion, but also that the inner pill material has also been digested. This application is particularly useful in experimental pharmacological settings. The identifier of these embodiments is one that does not necessarily emit a signal, but which can be optically inspected, e.g., visually or machine read, to obtain information about the composition with which it was associated prior to administration.

While the identifier may be an identifier that does not emit a signal, in certain embodiments (as summarized above) the identifier is one that does emit a signal. Depending on the needs of a particular application, the signal may be a generic signal, e.g., a signal that merely identifies that the composition has contacted the target site, or a unique signal, e.g., a signal which in some way uniquely identifies that a particular composition from a group or plurality of different compositions in a batch has contacted a target physiological site. As such, the identifier may be one that, when employed in a batch of unit dosages, e.g., a batch of tablets, emits a signal which cannot be distinguished from the signal emitted by the identifier of any other unit dosage member of the batch. In yet other embodiments, the identifier emits a signal that uniquely identifies a given unit dosage, even from other identical unit dosages in a given batch. Accordingly, in certain embodiments the identifier emits a unique signal that distinguishes a given type of unit dosage from other types of unit dosages, e.g., a given medication from other types of medications. In certain embodiments, the identifier emits a unique signal that distinguishes a given unit dosage from other unit dosages of a defined population of unit dosages, e.g., a prescription, a batch or a lifetime production run of dosage formulations. In certain embodiments, the identifier emits a signal that is unique, i.e., distinguishable, from a signal emitted by any other dosage formulation ever produced, where such a signal may be viewed as a universally unique signal (e.g., analogous to a human fingerprint which is distinct from any other fingerprint of any other individual and therefore uniquely identifies an individual on a universal level). In one embodiment, the signal may either directly convey information about the composition, or provide an identifying code, which may be used to retrieve information about the composition from a database, i.e., a database linking identifying codes with compositions.

The identifier may be any component or device that is capable of generating a detectable signal following activation in response to a stimulus. In certain embodiments, the stimulus activates the identifier to emit a signal once the composition comes into contact with a physiological target site, e.g., as summarized above. For example, a patient may ingest a pill that upon contact with the stomach fluids, generates a detectable signal. Depending on the embodiment, the target physiological site or location may vary, where representative target physiological sites of interest include, but are not limited to: a location in the gastrointestinal tract (such as the mouth, esophagus, stomach, small intestine, large intestine, etc.); another location inside the body, such as a parental location, vascular location, etc.; or a topical location; etc.

In certain embodiments the stimulus that activates the identifier is an interrogation signal, such as a scan or other type of interrogation. In these embodiments, the stimulus activates the identifier, thereby emitting a signal which is then received and processed, e.g., to identify the composition in some manner.

In certain of these embodiments, the identifier may include a power source that transduces broadcast power and a signal generating element that modulates the amount of transduced power, such that a signal is not emitted from the identifier but instead the amount of broadcast power transduced by the identifier is detected and employed as the “signal.” Such embodiments are useful in a variety of applications, such as applications where the history of a given composition is of interest.

In certain embodiments, the identifier is dimensioned to be complexed with the active agent/pharmaceutically acceptable carrier component of the composition so as to produce a composition that can be readily administered to a subject in need thereof. As such, in certain embodiments, the identifier element is dimensioned to have a width ranging from about 0.05 mm to about 1 mm, such as from about 0.1 mm to about 0.2 mm; a length ranging from about 0.05 mm to about 1 mm, such as from about 0.1 mm to about 0.2 mm and a height ranging from about 0.1 mm to about 1 mm, such as from about 0.05 mm to about 0.3 mm, including from about 0.1 mm to about 0.2 mm. In certain embodiments the identifier is 1 mm3 or smaller, such as 0.1 mm3 or smaller, including 0.2 mm3 or smaller. The identifier element may take a variety of different configurations, such as but not limited to: a chip configuration, a cylinder configuration, a spherical configuration, a disc configuration, etc, where a particular configuration may be selected based on intended application, method of manufacture, etc.

The signal generation component of the identifier element is a structure that, upon activation by the activation component, emits a detectable signal, e.g., that can be received by a receiver. The signal generation component of certain embodiments can be any convenient device that is capable of producing a detectable signal and/or modulating transduced broadcast power, upon activation by the activation component. Detectable signals of interest include, but are not limited to: conductive signals, acoustic signals, etc. The signals emitted by the signal generator may be generic or unique signals, where representative types of signals of interest include, but are not limited to: frequency shift coded signals; amplitude modulation signals; frequency modulation signals; etc.

In certain embodiments, methods include modulating a therapeutic regimen based data obtained from the compositions. For example, data may be obtained which includes information about patient compliance with a prescribed therapeutic regimen. This data, with or without additional physiological data, e.g., obtained using one or more sensors, such as the sensor devices described above, may be employed, e.g., with appropriate decision tools as desired, to make determinations of whether a given treatment regimen should be maintained or modified in some way, e. g., by modification of a medication regimen and/or implant activity regimen. As such, methods of invention include methods in which a therapeutic regimen is modified based on signals obtained from the composition(s).

The present invention provides the clinician an important new tool in their therapeutic armamentarium: automatic detection and identification of pharmaceutical agents actually delivered into the body. The applications of this new information device and system are multi-fold. Applications include, but are not limited to: (1) monitoring patient compliance with prescribed therapeutic regimens; (2) tailoring therapeutic regimens based on patient compliance; (3) monitoring patient compliance in clinical trials; (4) monitoring usage of controlled substances; and the like.

One type of application in which the subject compositions and systems find use is in monitoring patient compliance with prescribed therapeutic regimens. By monitoring patient compliance is meant tracking whether a patient is actually taking medication in the manner prescribed to the patient. As such, the present invention provides accurate data of when a pill has been taken and which pill has been taken. This allows the precise determination of which pill was taken at a specific point in time. Such monitoring capability assures patients are taking the prescribed medication correctly. This information avoids the potential for over prescription of medications that are not actually being taken. By example, if pain killers are intended to be administered to a patient, it is possible to verify with the present invention that the patient did in fact take those painkillers in a certain period of time. This knowledge is an important tool in limiting the illicit sale of unconsumed drugs to an unintended party. In the case of cardio vascular pills, the clinician or care giver is able to verify that the amount of the drug was taken has been taken at approximately the right point and time. Thus, the true efficacy of the drug can be accurately evaluated. Proper administration and patient compliance is especially critical in Alzheimer's, psychiatric, and alcohol aversion drugs, and in the treatment of rest home residents. In the case of accidental and other overdoses situations, the intervening clinician will be able to discern how far the ingestion has proceeded, and how many pills are involved.

In embodiments of the present invention, correct, timely ingestion of the drugs will automatically trigger a prescription refill signal which is forwarded to a pharmacy data system, and in some cases the refill will be automatically delivered directly to the patient's home, or released by a device in the patient's home some period of time later. This feature is particularly valuable in patients with compromised mental capacity and/or limited physical mobility.

The invention is particularly useful in complex administration regimens, such as when multiple pharmaceuticals are being taken, and confusion is more likely to occur. The inventive pills can have multiple external layers, with only correct dosage allowing dissolution and absorption of the pharmaceutical component. Specific indicators, such as electrical conduction velocity in the heart or electrolytic levels in the blood in response to pharmaceutical can also be titrated.

In certain embodiments, a patient can be alerted when the patient is in some way non-compliant with a given treatment regimen. For example, by a sound, visual, or computer reminder, if the pharmacological regimen is not being accurately adhered to, a reminder is provided. If that reminder is not accurately responded to, the system can provide an alert to family members, caregivers, or clinicians in order to remedy the gap in treatment or overdose. The device may also automatically modify the dosage and timing of the regimen to compensate for prior non-standard dosing.

The gaming systems include any hardware device, software, and/or communications component, as well as systems and subsystems of the same, which generally function to provide a service or activity related to the physiologic data. The gaming systems, for example, collect, manipulate, calculate, transmit, receive, store, utilize and/or communicate at least a portion of the physiologic data for use with a game or recreational activity. The gaming system, for example, is selected from a group consisting essentially of: software games; interactive games; device games; and online games.

Each gaming system may be built around predefined function(s) or service(s) and may directly receive physiologic data via the hub. Alternatively, the gaming system may indirectly receive, i.e., via another system, computer, etc., the physiologic data. In certain aspects, the gaming system and the hub may be an integral or overlapping system, e.g., mobile telephone having software application(s) that enable both receipt of the physiologic data from the personal signal receiver and facilitate a gaming activity that utilizes the physiologic data.

Each of the foregoing aspects is described in greater detail hereinafter.

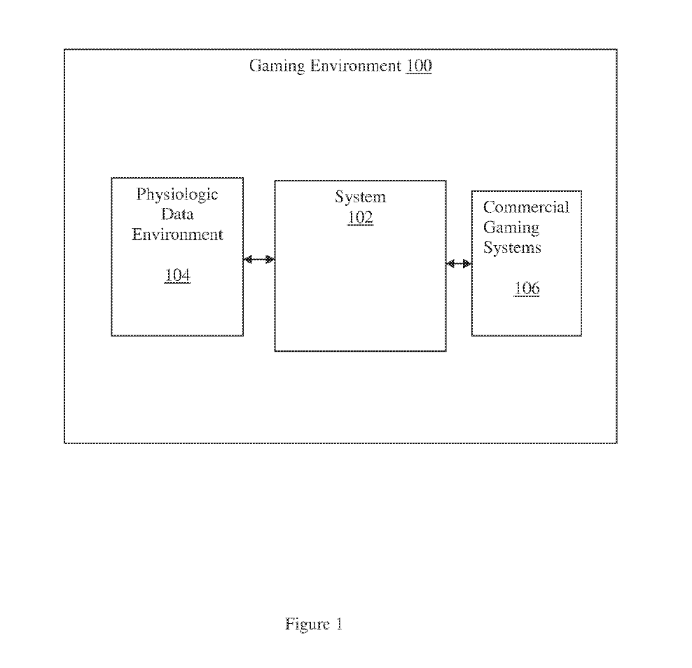

FIG. 1 provides a diagrammatic representation of a gaming environment 100. The gaming environment 100 may include, for example, a system for incorporating physiologic data 102, a physiologic data environment 104, and one or more commercial gaming systems 106.

More particularly, the gaming environment 100 includes any environment having therein, or associated with, data or communication of physiologic data for a gaming or recreational purpose. Communication includes any method, act, or vehicle of communication, and/or combinations thereof. For example, communication methods include manual, wired, and wireless, etc. Wireless technologies include radio signals, such as x-rays, ultraviolet light, the visible spectrum, infrared, microwaves, and radio waves, etc. Wireless services include voice and messaging, handheld and other Internet-enabled devices, data networking, etc.

Vehicles of communication include the Internet, wired channels, wireless channels, communication devices including telephones, computers, wire, radio, optical or other electromagnetic channels, and combinations thereof, including other devices and/or components capable of/associated with communicating data. For example, the communication environments include in-body communications, various devices, various modes of communications such as wireless communications, wired communications, and combinations of the same, etc.

In-body communications include any communication of data or information via the body, i.e., communication via or associated with inter-body aspects, intra-body aspects, and a combination of the same. For example, inter-body aspects include communications associated with devices designed to attach to a body surface. Intra-body aspects include communications associated with data generated from within the body, e.g., by the body itself or by a device implanted, ingested, or otherwise locatable in, or partially in, the body. For example, intra-body communications are disclosed in the U.S. Provisional Patent No. 61/251088, the entire content of which is hereby incorporated by reference.

Communications include and/or may be associated with software, hardware, circuitry, various devices, and combinations thereof.

The devices include devices associated with physiologic data generation, transmission, reception, communication, etc. The devices further include various implantable, ingestible, insertable, and/or attachable devices associated with the human body or other living organisms. The devices still further include multimedia devices such as telephones, stereos, audio players, PDA's, handheld devices, and multimedia players.

Wireless communication modes include any mode of communication between points that utilizes, at least in part, wireless technology including various protocols and combinations of protocols associated with wireless transmission, data, and devices. The points include, for example, wireless devices such as wireless headsets, audio and multimedia devices and equipment, such as audio players and multimedia players, telephones, including mobile telephones and cordless telephones, and computers and computer-related devices and components, such as printers.

Wired communication modes include any mode of communication between points that utilizes wired technology including various protocols and combinations of protocols associated with wired transmission, data, and devices. The points include, for example, devices such as audio and multimedia devices and equipment, such as audio players and multimedia players, telephones, including mobile telephones and cordless telephones, and computers and computer-related devices and components, such as printers.

The system for incorporating physiologic data 102 enables exchange, transmission, receipt, manipulation, management, storage, and other activities and events related to physiologic data. Such activities and events may be contained within the system for incorporating physiologic data 102, partially integrated with the system for incorporating physiologic data 102, or associated with externalities, e.g., activities, systems, components, and the like which are external to the system for incorporating physiologic data 102. Externalities include, for example, the gaming environment 100 and commercial gaming systems 106, either or both of which may also be integral to, or partially integrated with, the system for incorporating physiologic data 102.

The physiologic data environment 104 includes any source of information or data, including remote computer systems, local computer devices, etc. The information or data may comprise physiologic data in whole or in part, e.g., aggregated or generated with other types of data. The physiologic data may be pure or refined, e.g., physiologic data from which inferences are drawn.

The commercial gaming systems 106 include various existing systems that utilize one or various types of data to accomplish a particular gaming or recreational purpose. One example of the commercial gaming system 106 is an online poker game. Another example of the commercial gaming system is a tennis game having an electronic scoreboard. Still another example of the commercial gaming system is a handheld electronic gaming device.

The system for incorporating physiologic data 102, the physiologic data environment 104, and the commercial gaming systems 106 are discussed in greater detail hereinafter.

### 2.0 System for Incorporating Physiologic Data in a Gaming Environment

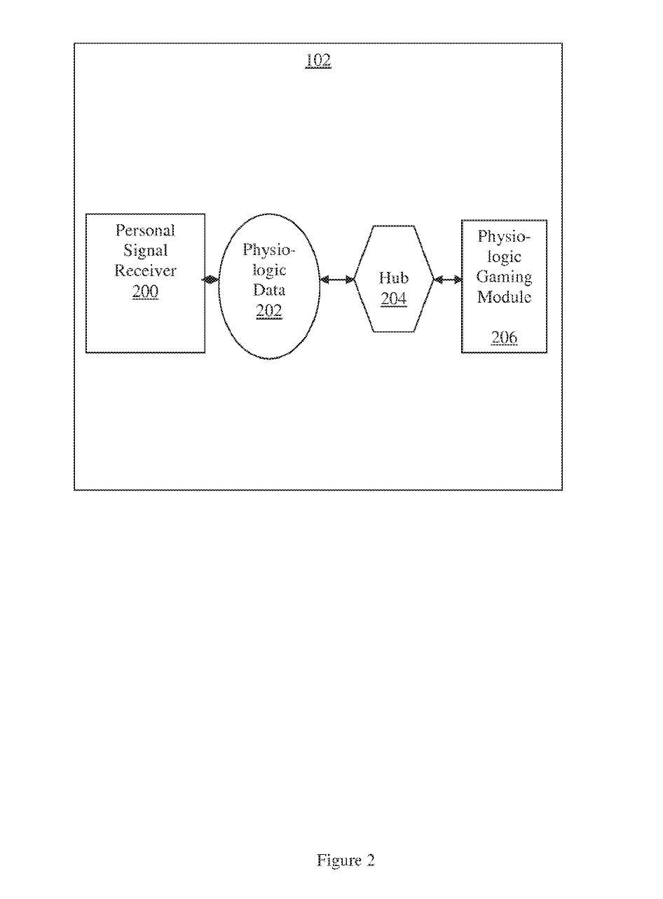

FIG. 2 provides an exemplary diagrammatic representation of the system for incorporating physiologic data 102 in the gaming environment 100 of FIG. 1. The system for incorporating physiologic data 102 includes a personal signal receiver 200, physiologic data 202, a hub 204, and a physiologic gaming module 204.

### Personal Signal Receiver

The personal signal receiver 200 includes any device, alone or in combination with other components, devices, systems, etc., that facilitate communication of the physiologic data 202. The personal signal receiver includes devices such as those disclosed and described in U.S. patent application Ser. No. 11/912,475 entitled “Pharma Informatics Systems”, filed Oct. 24, 2007; the PCT Patent Application Serial No. PCT/US2007/10688 entitled “Patient Customized Therapeutic Regimens”, filed May 2, 2007; The PCT Patent Application Serial No. PCT/US2007/015547 entitled “Smart Parenteral Systems”, filed Jul. 6, 2007; the PCT Patent Application Serial No. PCT/US2007/022257 entitled “In vivo Low Voltage Oscillator Medical Devices,” filed Oct. 17, 2007; the PCT Patent Application Serial No. PCT/US07/24225 entitled, “Active Signal Processing Personal Health Signal Receivers”, filed Nov. 19, 2007; the PCT Patent Application Serial No. US2008/52845 entitled, “Ingestible Event Marker Systems”, filed Feb. 1, 2008; and the U.S. Patent Application Serial No. 61/079,082 entitled “Ingestible Event Marker Data Framework”, filed Jul. 8, 2008. Each of the foregoing is incorporated herein in its entirety by reference.

In certain embodiments, the systems include an external device which is distinct from the receiver (which may be implanted or topically applied in certain embodiments), where this external device provides a number of functionalities. Such an apparatus can include the capacity to provide feedback and appropriate clinical regulation to the patient. Such a device can take any of a number of forms. By example, the device can be configured to sit on the bed next to the patient. The device can read out the information described in more detail in other sections of the subject patent application, both from pharmaceutical ingestion reporting and from psychological sensing devices, such as is produced internally by a pacemaker device or a dedicated implant for detection of the pill. The purpose of the external apparatus is to get the data out of the patient and into an external device. One feature of external apparatus is its ability to provide pharmacologic and physiologic information in a form that can be transmitted through a transmission medium, such as a telephone line, to a remote location such as a clinician or to a central monitoring agency.

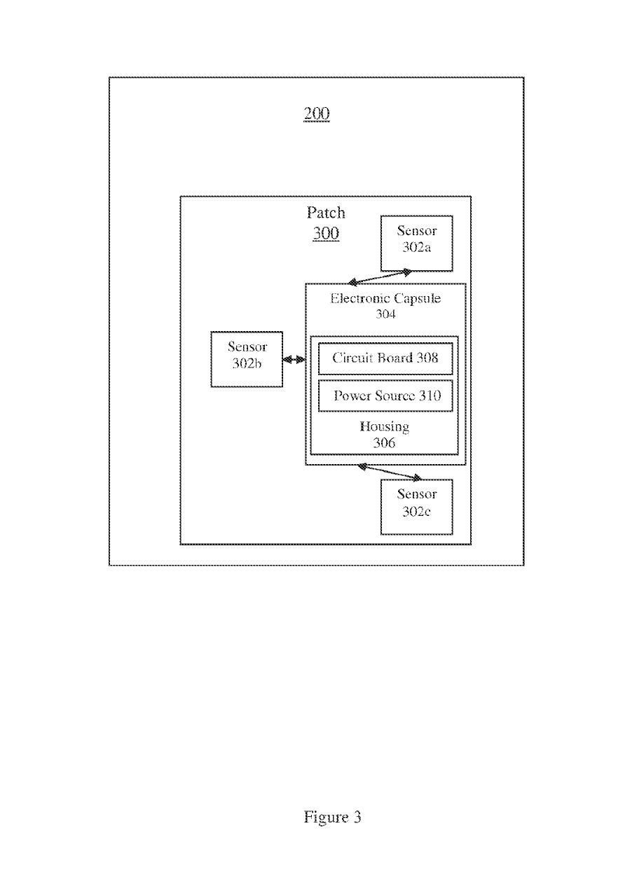

FIG. 3 illustrates the exemplary personal signal receiver 200 of the system for incorporating physiologic data in the gaming environment 100 of FIG. 2. In various aspects, the personal signal receiver 200 includes a patch 300; one or more sensors 302, e.g., sensors 302a-c, connected to the patch 300; and an electronics capsule 304 in electronic communication with the sensors 302.

The patch 300 may incorporate various form factors and materials. In one example, the patch may be similar in design, shape, size, and material to an adhesive bandage, i.e., may be removably-attachable to a person's person. Specific dynamics of design, shape, design, and material may vary according to use, environment, placement, etc. For example, the design may incorporate various constructs and patterns. The constructs, for example, may include one or more layers, e.g., a substrate having an adhesive layer. The patterns may include various aesthetic features, e.g., various patterns, various colors, decals, imprints, etc., and non-aesthetic features, e.g., breathable materials, non-allergenic materials, etc. The shape may vary, e.g., oval, circular, triangular, kidney-shaped, rectangular, etc. The size may vary, e.g., approximately 75 mm. by 120 mm., approximately 50 mm. by 80 mm., approximately 25 mm. by 40 mm., or other sizes, as desired. The material may incorporate any material or combinations of materials capable of carrying out the functionality of the patch as herein described. Further, in various aspects, considerations may be given to position, energy conservation schemes, carrier identification, decoding and error correcting.

The sensors 302 typically contact the person's person, e.g., are removably attached to the torso. In various aspects, the sensors 302 may be removably or permanently attached to the patch 300. For example, the sensors may be removably connected to the patch 300 by snapping metal studs. The sensors 302 may comprise, for example, various devices capable of sensing or receiving the physiologic data 202. The types of sensors include, for example, electrodes such as biocompatible electrodes. The sensors may be configured, for example, as a pressure sensor, a motion sensor, an accelerometer, an electromyography (EMG) sensor, an ingestible event marker, a biopotential sensor, an electrocardiogram sensor, a temperature sensor, a tactile event marker sensor, and an impedance sensor.

The electronics capsule 304 communicates with the sensors and/or other devices. In various aspects, for example, the electronics capsule 304 generates electronic communication with the sensors 302. In one example, the electronics capsule 304 includes a housing 306 having a circuit board 308 having electronic integrated circuits (not shown); and a power source 310. In various aspects, the housing 306 may include various features, e.g., watertight, hermetically sealed, etc. The circuit board having electronic circuits may electronically communicate with the sensors 302. The power source 310 may, for example, be a rechargeable power source such as a rechargeable battery.

In various aspects, different energy conservation schemes may be considered. Such schemes include a periodic wake-up, e.g., sensors and/or other components wake-up periodically such that energy, e.g., power source 310, is conserved during non-awake periods.

In various aspects, the personal signal receiver 200 may accomplish one or more of sensing functions using a signal receiving element, e.g., using electrodes of the personal signal receiver 200 for signal receiving and sensing applications, or the personal signal receiver 200 may include one or more distinct sensing elements that are different from the signal receiving element. The number of distinct sensing elements that may be present on (or at least coupled to) the signal receiver may vary, and may be one or more, two or more, three or more, four or more, five or more, ten or more, etc.

In various aspects, the personal signal receiver 200 may include a pair of electrodes that provide for dual functions of signal receiving and sensing. For example, in addition to receiving signal, the electrodes can also serve additional sensing functions. In certain aspects, the electrodes may be used to generate an IEGM (intra cardiac electrogram) at whatever site they are located. From that data, there are many kinds of processing that can be done, e.g., to detect various cardiac events, such as tachycardia, fibrillations, heart rate, etc. Another sensing capability that may be accomplished with two electrodes of the signal receiver employs measuring the impedance between the electrodes. The measured impedance will have some component which is determined by the trans-thoracic impedance, which relates to respiration. In this manner, the impedance data can be employed to obtain the respiratory rate of the subject. The electrodes may also be employed as sensors of fluid state of subject.

As mentioned above, one or more additional physiologic sensors distinct from the electrodes may be included in the signal receiver. For example, a temperature sensor, e.g., a thermistor, may be included in the signal receiver. If really precise temperature measurement are desired, there are other techniques like resistive temperature devices (RTDs), made out of platinum generally, which can give very precise measurements of temperature. An additional physiological sensor may include an LED and a photodiode combined into a pulse-oximeter, which may be employed to measure blood oxygenation, which would also give information about pulse pressure.

In addition, the personal signal receivers 200 may include a pressure sensor, e.g., where the signal receiver is implanted next to an artery to get measurements of arterial blood pressure. For example, one can get the pressure inside the body by putting a pressure sensitive membrane on the surface of the signal receiver. To get a more useful type of pressure, one usually wants to measure the venous or arterial blood pressure. In such a case, the membrane may be located in proximity to either an artery or a vein, so that as the artery pulsed it would exert a pressure on the pressure sensor. That could be calibrated to give an absolute pressure reading. Another possibility would be some sort of outrigger cuff, e.g., which cuffed around the artery. It could have strain gauges in it to measure pressure deflections, which would then be attached to the signal receiver.

Generally, the personal signal receivers 200 may also include analyte detection sensors. For example, specific chemical sensors may be incorporated into the signal receivers to detect the presence of various agents, e.g., glucose, BNP (B-type Natriuretic, which is associated with cardiac disease), etc. There are other ways that one could build an oxygen sensor, including selectively porous impedance cells, where the oxygen changes the pH of a cell, and then the conductivity of that is measured. Where the signal receiver includes an analyte detecting sensing element, this sensing element can be configured in the signal receiver in a number of different ways. For example, a sensor that includes a selectively permeable membrane which is permeable to the agent to be detected may be provided, where there is an isolated cell behind it, and the agent passes through the membrane, and changes the properties, usually electrical properties, of the cell, which are then measured. For example, there may be a small reservoir on the side of the signal receiver with a membrane across it, and the measuring electrical circuitry behind it. Another way of detecting agents employs sensors known in the art as ChemFET sensors, which are based on the binding of analyte to the sensor causing a change in the conductivity. There may be included a material with electrical properties (or other properties) that are changed when the material binds to it. Various proteins may be detected that way.

### Physiologic Data

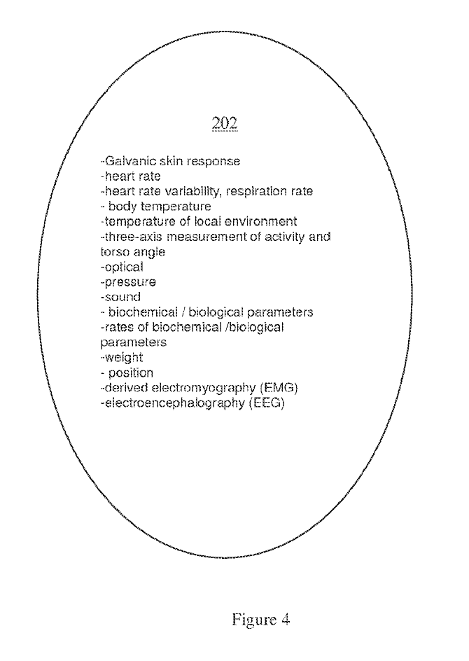

FIG. 4 illustrates exemplary physiologic data associated with the system for incorporating physiologic data in a gaming environment 102 of FIG. 2. The physiologic data 200 include data associated physiologic events, parameters, measurements, etc. Such data include, for example, Galvanic skin response, heart rate, heart rate variability, respiration rate, body temperature, temperature of local environment, three-axis measurement of activity and torso angle, optical, pressure, sound, biochemical/biological, weight, position, derived electromyography (EMG), and electroencephalography (EEG). The physiologic data further include those data set out in the U.S. patent application Ser. Nos. 10/734,490; 10/764,429; 10/764,127; 10/764,125; 11/025,657; 11/324,196; 11/664,340; 11/731,786; 11/718,201; 11/897,942; 11/912,475; 12/063,097; 12/063,095; as well as PCT Application Serial Nos: PCT/ US2007/015547; and PCT/US2008/52845, each of which is incorporated herein in its entirety by reference.

### The Hub

The hub 204 includes any hardware, software, and/or communications component(s) in any combination/configuration, which generally function to communicate the physiologic data 202. One example includes communicating the physiologic data 202 to the physiologic gaming module 206.

In various aspects, the hub 204 receives, generates, communicates, and/or transmits, the physiologic data 202, alone or in combination with other data, i.e., non-physiologic data from various sources. Communication from the hub 204 includes any transmission means or carriers, and combinations thereof, including wireless, wired, RF, conductive, etc. as is known in the art or as may become available in the future.

The hub 204 comprises various categories of devices, e.g., personal communication devices, base stations, and mobile telephones.

The systems are reviewed in terms of communication between an identifier on a pharmaceutical composition and a receiver. However, the systems are not so limited. In a broader sense, the systems are composed of two or more different modules that communicate with each other, e.g., using the transmitter/receiver functionalities as reviewed above, e.g., using the monopole transmitter (e.g., antenna) structures as described above. As such, the identifier elements may be incorporated into any of a plurality of different devices, e.g., to provide a communications system between two self-powered devices in the body, where the self-powered devices may be sensors, data receivers and storage elements, effectors, etc. In an exemplary system, one of these devices may be a sensor and the other may be a communication hub for communication to the outside world. This embodiment may take a number of forms. There can be many sensors, many senders and one receiver. They can be transceivers so both of these can take turns sending and receiving according to known communication protocols. In certain embodiments, the means of communication between the two or more individual devices is the mono polar system, e.g., as described above. In these embodiments, each of these senders may be configured to take turns sending a high frequency signal into the body using a monopole pulling charge into and out of the body which is a large capacitor and a conductor. The receiver, a monopole receiver is detecting at that frequency the charge going into and out of the body and decoding an encrypted signal such as an amplitude modulated signal or frequency modulated signal. This embodiment of the present invention has broad uses. For example, multiple sensors can be placed and implanted on various parts of the body that measure position or acceleration. Without having wires connecting to a central hub, they can communicate that information through a communication medium.

Personal communication devices include, for example, devices having communication and computer functionality and typically intended for individual use, e.g., mobile computers, sometimes referred to as “handheld devices”.

Base stations comprise any device or appliance capable of receiving data such as physiologic data. Examples include computers, such as desktop computers and laptop computers, and intelligent devices/appliances.

Intelligent devices/appliances include consumer and home devices and appliances that are capable of receipt of data such as physiologic data. Intelligent devices/appliances may also perform other data-related functions, e.g., transmit, display, store, and/or process data. Examples of intelligent devices/appliances include refrigerators, weight scales, toilets, televisions, door frame activity monitors, bedside monitors, bed scales. Such devices and appliances may include additional functionality such as sensing or monitoring various physiologic data, e.g., weight, heart rate, etc. Mobile telephones include telephonic communication devices associated with various mobile technologies, e.g., cellular networks.

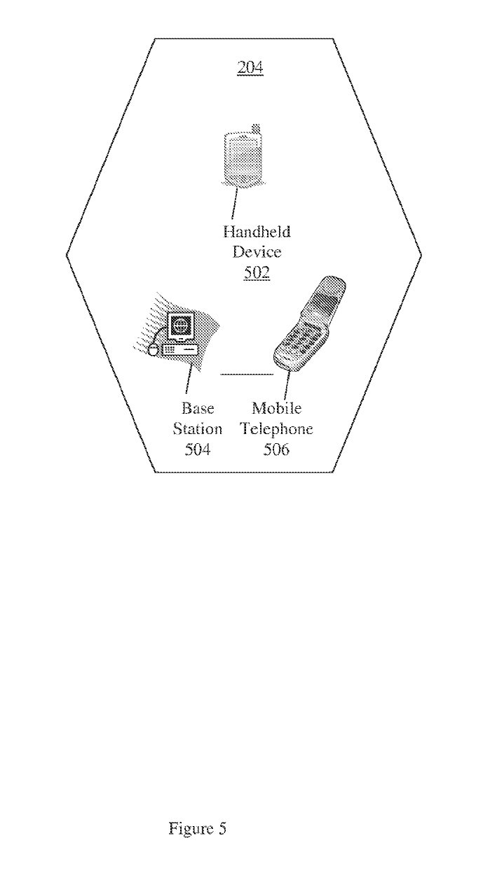

FIG. 5 illustrates a hub associated with the system for incorporating physiologic parameters of FIG. 2. In one aspect, the hub 204 includes a personal communication device, such as a handheld device 502; a base station 504; and a mobile telephone 506.

The handheld device 502, also referred to as a “mobile computer”, includes, for example, computing devices having computer-related functionality, e.g., typically having a display screen with touch input functionality, a miniature keyboard, etc. Types of handheld devices include, for example, a personal digital assistant (PDA) having the input and output combined into a touch-screen interface; and enterprise digital assistants offering integrated data capture devices like bar code, radio frequency identification (RFID), and smart card readers, etc.

In various aspects, the handheld device 502 includes software, e.g., a software agent/application, associated with the physiologic data 202. In various aspects of the handheld device 502, the software is preconfigured, i.e., configurable by the manufacturer/retailer; configurable by the consumer, i.e., downloadable from a website; or a combination of the same.

One example of software is a software agent for interaction with an online game.

The base station 504 includes systems, subsystems, devices, and/or components that receive, transmit, and/or relay the physiologic data 202. In various aspects, the base station 504 communicably interoperates with a receiver such as the personal signal receiver 200 and a communications network such as the Internet. Examples of base stations 404 are computers, e.g., servers, personal computers, desktop computers, laptop computers, intelligent devices/appliances, etc., as heretofore discussed.

In various aspects, the base station 504 may be embodied as an integrated unit or as distributed components, e.g., a desktop computer and a mobile telephone in communication with one another and in communication with a patch receiver and the Internet.

In various aspects, the base station 504 includes the functionality to wirelessly receive and/or wirelessly transmit data, e.g., physiologic data 202 received from and transmitted to the patch receiver 400 and the Internet.

Further, in various aspects, the base station 504 may incorporate and/or be associated with, e.g., communicate with, various devices. Such devices may generate, receive, and/or communicate data, e.g., physiologic data 202. The devices include, for example, “intelligent” devices such as gaming devices, e.g., electronic slot machines, handheld electronic games, electronic components associated with games and recreational activities, etc.

The mobile telephone 506 includes, for example, devices such as a short-range, portable electronic device used for mobile voice or data communication over a network of specialized cell site base stations. The mobile telephone 506 is sometimes known as or referred to as “mobile”, “wireless”, “cellular phone”, “cell phone”, or “hand phone (HP)”.

In addition to the standard voice function of a telephone, various aspects of mobile telephones may support many additional services and accessories such as short message service (SMS) for text messaging, email, packet switching for access to the Internet, java gaming, wireless, e.g., short range data/voice communications, infrared, camera with video recorder, and multimedia messaging system (MMS) for sending and receiving photos and video. Some aspects of mobile telephones connect to a cellular network of base stations (cell sites), which is, in turn, interconnected to the public switched telephone network (PSTN) or satellite communications in the case of satellite phones. Various aspects of mobile telephones can connect to the Internet, at least a portion of which can be navigated using the mobile telephones.

In various aspects, the mobile telephone 506 includes software, e.g., a software agent/application, associated with the physiologic data 202. One example is an auto refill application related to or integrated with an auto refill system to facilitate automated prescription refill functions. In various aspects of the mobile telephone 506, the software is preconfigured, i.e., configurable by the manufacturer/retailer; configurable by the consumer, i.e., downloadable from a website; or a combination of the same.

Further, various aspects of the hub include combinations of devices. One such combination is the personal signal receiver 200 in communication with the handheld device 502 or the mobile telephone 506. Thus, for example, the personal signal receiver 200 wirelessly transmits physiologic data 202 to the mobile telephone 506 having a receiver and a software agent available thereon. The receiver of the mobile telephone 506 receives the physiologic data 202. A software agent, e.g., an application, processes the physiologic data 202 and displays various information related to the physiologic data 202 via, for example, a customized graphical user interface (GUI). In various aspects, the software agent generates displays with a predetermined “look and feel”, i.e., recognizable to a user as belonging to a predetermined group of software programs, GUIs, source devices, communities, gaming software, etc.

### The Physiologic Gaming Module

The physiologic gaming module includes any hardware, software, and/or communications component(s) in any combination/configuration, which generally includes physiologic data 202. In various aspects, the physiologic gaming module 206 may be standalone, e.g., a software module and/or associated and/or integrated with a device, including a hub device, e.g., the mobile telephone 506. In various aspects, the physiologic gaming module is associated with devices, software, systems, etc. in addition to the current invention, e.g., commercial gaming systems.

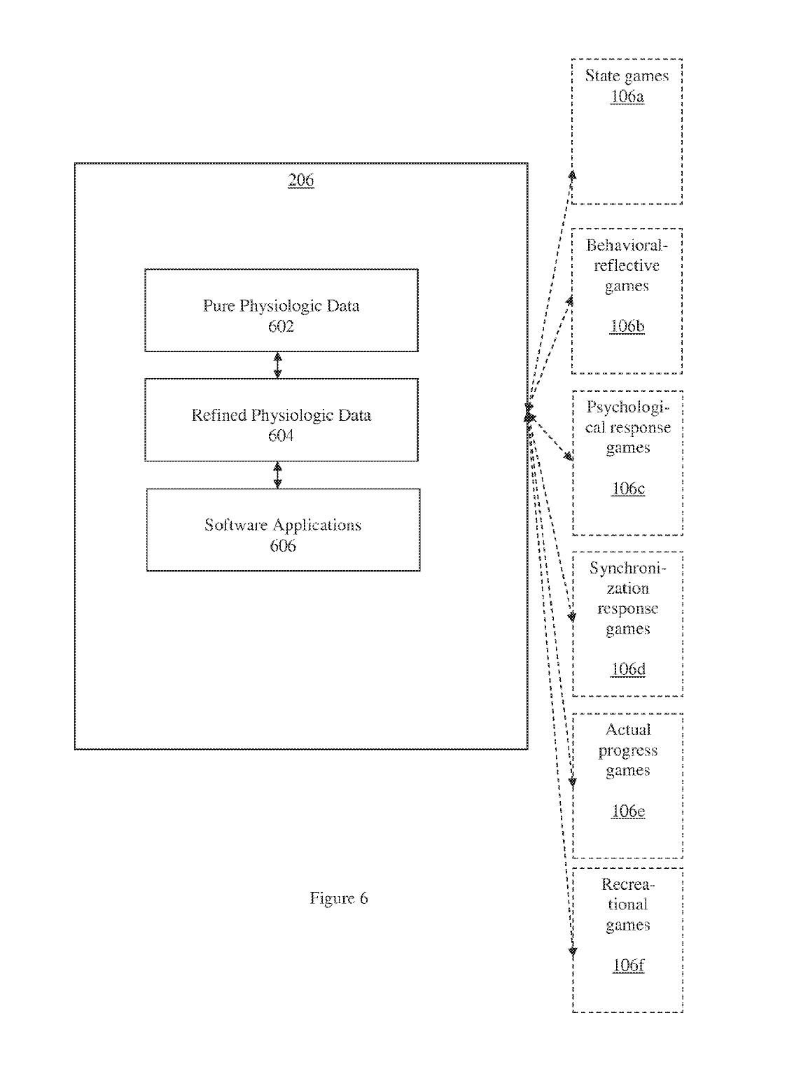

FIG. 6 illustrates a physiologic gaming module 206 associated with the system for incorporating the physiologic data 202 of FIG. 2. In various aspects, the physiologic module includes, for example, pure physiologic data 602, refined physiologic data 604, and/or one or more software applications 606.

The pure physiologic data 202 comprises the physiologic data derived via the personal signal receiver, e.g., heart rate, heart rate variability, etc. The refined physiologic data 604 includes other data, refined physiologic data, etc. For example, if the heart exceeds a predetermined range of normal, a gaming inference may be drawn that the individual is stressed/excited. The stressed/excited factor may be included into the game to decide a course of action, etc. The software applications comprise, for example, gaming software, software associated with interactive games, software associated with game-related devices, online games, etc.

In one scenario, the physiologic data may be used to reflect a state. For example, a particular participant's heart rate and heart rate variability are derived via the personal signal receiver. The heart rate and heart rate variability are transmitted via the hub in a physiologic gaming module 206 to an online, interactive state games 106a. The heart parameters are analyzed and an inference drawn that the particular heart parameters are indicative of a stressed and/or an excited state of a participant. The inferences may be translated and reflected in the displayed activity of the avatar associated with the particular participant, e.g., stress expression on avatar's face.

In another scenario, the physiologic data may be used to reflect behavior, e.g., active motion, etc. For example, the personal signal receiver may be attached to various body locations, e.g., both arms and both hands of the participant. The personal signal receiver measures body motion and angle, transmits the measurements via the hub in a physiologic gaming module 206 to a behavioral-reflective game 106b, e.g., the avatar associated with the participant reflects the same motions/behaviors detected by the personal signal receiver.

In still another scenario, the physiologic data may used to invoke, at least in part, a physiological response in the person in which the physiologic parameters are measured and/or in other participants. For example, a particular participant may engage in a game involving shooting a weapon. The particular participant's heartbeat is detected and reproduced as a sound effect in a physiologic gaming module 206 and presented to both the particular participant and to opponent participants. In psychological response games 106c, the audible level of the reproduced heartbeat may increase as the players' gaming avatars converge towards a single gaming location, thus triggering various psychological responses in the “stalked” opponents, e.g., fear, manifested as an increase in heart rate of the “stalked” opponents. This psychological response may then be detected and audibly reflected back to all the participants, in turn generating a psychological response of confidence, etc., in the “stalker”.

In yet another scenario, the physiologic data may be “mapped” between participants to synchronize, align, compare, etc., personal dynamics. For example, the movements and heartbeats of two participants are measured. In synchronization response games 106d, such as an online “dancing” game, the partners get points for synchronizing their moves, as provided in a physiologic gaming module 206 and as reflected in their avatars as well as for synchronizing their hearts to beat in time together.

In a further scenario, the physiologic data may be compared to the online game goals to indicated actual progress, etc. For example, a participant in an online “swim” competition holds his breath. His respiration is measured to determine how long he actually held his breath. The measurement is translated into a metric in a physiologic gaming module 206 of an actual progress game 106e that reflects how far the participant's avatar would have traveled underwater according to how long the participant actually held his breath.

In a still further scenario, the physiologic data may be reported or reflected in respect to actual recreation games 106f. For example, the heart rate of two tennis opponents may be detected via the personal signal receiver and transmitted via the hub in a physiologic gaming module 206 for reporting on a monitor, e.g., an electronic scoreboard. In another example, a football player's range of motion in attempting a field goal may be measured via personal signal receivers located on various positions on his leg and torso. The measurements may be transmitted, reported via a scoreboard, and compared to other players, etc.

In the foregoing, the term “games” includes pursuits and applications utilizing such physiologic data, e.g., warfare-related software applications; intrigue and other software or actual pursuits, etc.

One skilled in the art will recognize that the above-described scenarios may be embodied in various games, e.g., commercial gaming systems, directly in the physiologic gaming module, the hub, etc. Further, the above-described scenarios are illustrative only and in no way limiting to the invention described herein.

### 3.0 Method for Incorporating Physiologic Data in a Gaming Environment

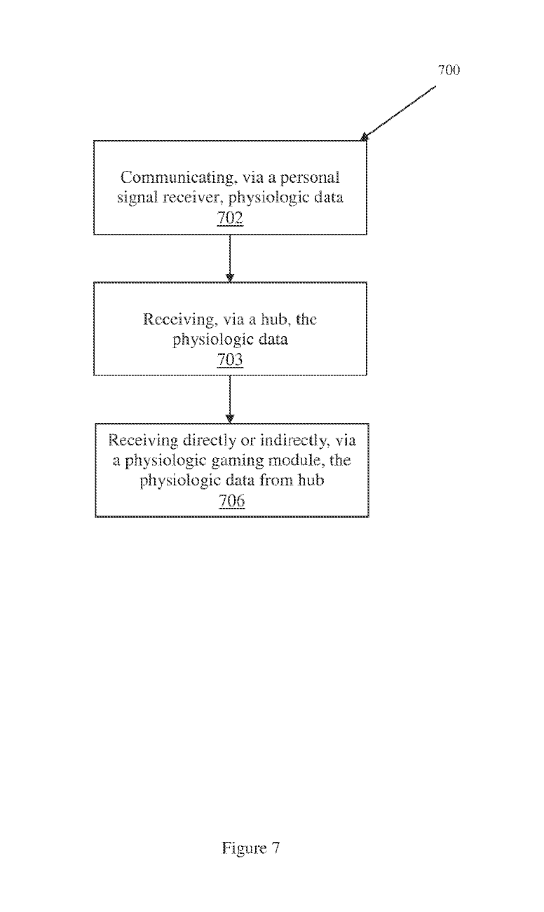

FIG. 7 illustrates a method for incorporating physiologic data in a gaming environment 700. The method includes steps of communicating, via a personal signal receiver, physiologic data at 702; receiving, via a hub, the physiologic data at 704; and receiving directly or indirectly, via a physiologic gaming module, the physiologic data from the hub at 706. A skilled artisan will recognize that various other aspects apply.

### 4.0 Apparatus for Incorporating Physiologic data in a Gaming

One aspect comprises, for example, a storage medium having instructions, that when executed by a computing platform, result in execution of a method of utilizing physiologic data in a gaming environment, comprising: communicating, via a personal signal receiver, physiologic data; receiving, via a hub, the physiologic data; and receiving directly or indirectly, via a physiologic gaming module, the physiologic data from the hub.

Further, aspects disclosed herein may be performed in a data processing system. To illustrate, a diagrammatic system comprises, for example, a processor, a main memory, a static memory, a bus, a video display, an alpha-numeric input device, a cursor control device, a drive unit, a signal generation device, a network interface device, a machine readable medium, instructions and a network, according to one aspect.

The diagrammatic system may indicate a personal computer and/or a data processing system in which one or more operations disclosed herein may be performed. The processor may be a microprocessor, a state machine, an application-specific integrated circuit, a field programmable gate array, etc. The main memory may be a dynamic random access memory and/or a primary memory of a computer system. The static memory may be a hard drive, a flash drive, and/or other memory information associated with the data processing system.

The bus may be an interconnection between various circuits and/or structures of the data processing system. The video display may provide graphical representation of information on the data processing system. The alpha-numeric input device may be a keypad, a keyboard and/or any other input device of text, e.g., a special device to aid the physically challenged. The cursor control device may be a pointing device such as a mouse. The drive unit may be a hard drive, a storage system, and/or other longer term storage subsystem. The signal generation device may be a bios and/or a functional operating system of the data processing system. The network interface device may be a device that may perform interface functions such as code conversion, protocol conversion and/or buffering required for communication to and from the network. The machine readable medium may provide instructions on which any of the methods disclosed herein may be performed. The instructions may provide source code and/or data code to the processor to enable any one/or more operations disclosed herein.

Although the present aspects have been described with reference to specific example aspects, it will be evident that various modifications and changes may be made to these aspects without departing from the broader spirit and scope of the various aspects. For example, the various devices, modules, etc. described herein may be enabled and operated using hardware circuitry, e.g., CMOS based logic circuitry, firmware, software and/or any combination of hardware, firmware, and/or software, e.g., embodied in a machine readable medium.

For example, the various electrical structure and methods may be embodied using transistors, logic gates, and electrical circuits, e.g., Application Specific Integrated circuitry (ASIC) and/or in Digital Signal Processor (DSP) circuitry. For example, the receive module and the communicate module and other modules may be enabled using one or more of the technologies described herein.

In addition, it will be appreciated that the various operations, processes, and methods disclosed herein may be embodied in a machine-readable medium and/or a machine accessible medium compatible with a data processing system, e.g., a computer system, and may be performed in any order. Accordingly, the specification and drawings are to be regarded in an illustrative rather than a restrictive sense.

Any or all data associated with the aforementioned devices and methods, for example, may be used alone or in combination with other data to constitute physiologic data, i.e., data having an physiologic aspect.

In certain aspects, the system and/or method steps further include/utilize an element for storing data, i.e., a data storage element, where this element is present on an external device, such as a sports device, PDA, smart phone, computer server, etc. Typically, the data storage element is a computer readable medium. The term “computer readable medium” as used herein refers to any storage or transmission medium that participates in providing instructions and/or data to a computer for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external to the computer. A file containing information may be “stored” on a computer readable medium, where “storing” means recording information such that it is accessible and retrievable at a later data by a computer and/or computer-related component. With respect to computer readable media, “permanent memory” refers to memory that is permanent. Permanent memory is not erased by termination of the electrical supply to a computer of processor. Computer hard-drive ROM, i.e., not used as virtual memory, CD-ROM, floppy disk and DVD are all examples of permanent memory. Random Access Memory (RAM) is an example of non-permanent memory. A file in permanent memory may be editable and re-writable.

Also provided are computer executable instructions, i.e., programming, for performing the above methods, e.g., for programming the personal signal receiver and other components of the system. The computer-executable instructions are present on a computer readable medium. Accordingly, various aspects provide a computer readable medium containing programming for use in providing ingestible event marker data.

As such, in certain aspects the systems include one or more of: a data storage element, a data processing element, a data display element, a data transmission element, a notification mechanism, and a user interface. These elements may be present or otherwise associated with at least one of the physiologic data, the hub, and the physiologic gaming module.

One of the above-described systems is reviewed in terms of a receive module and a communicate module. The aspects, however, are not so limited. In a broader sense, the systems are composed of two or more different modules that communicate with each other, e.g., using the hub functionalities as reviewed above, e.g., using the physiologic data in the communication, e.g., using the physiologic gaming module's functionality.

It is to be understood that this invention is not limited to particular aspects described, and as such may vary. It is also to be understood that the terminology used herein is for the purpose of describing particular aspects only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual aspects described and illustrated herein has discrete components and features which may be readily separated from or combined with the features of any of the other several aspects without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and aspects of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary aspects shown and described herein. Rather, the scope and spirit of present invention is embodied by the appended claims.

What is claimed is:

1. An apparatus, comprising:
a gaming module implemented in at least one of a memory or a processing device, the gaming module configured to receive patient compliance data associated with compliance of a patient with a therapeutic regimen, the gaming module configured to modify an aspect of an electronic game of the patient based on the patient compliance data to produce modified game data, the gaming module configured to send a signal associated with the modified game data such that the modified game data is presented to the patient.

2. The apparatus of claim 1, further comprising a hub configured to generate the patient compliance data, the gaming module configured to receive the patient compliance data from the hub.

3. The apparatus of claim 1, further comprising a hub configured to generate the patient compliance data in response to receiving an indication of ingestion of an ingestible event marker by the patient as part of the therapeutic regimen, the gaming module configured to receive the patient compliance data from the hub.

4. The apparatus of claim 1, further comprising a hub configured to receive, from a receiver associated with the patient, an indication generated by the receiver upon detection of ingestion of an ingestible event marker by the patient as part of the therapeutic regimen, the hub configured to generate the patient compliance data based on the indication.

5. The apparatus of claim 1, wherein the gaming module is within a device, the gaming module configured to receive the patient compliance data from a hub (1) different from the device and (2) that generates the patient compliance data.

6. The apparatus of claim 1, wherein the gaming module is within a device, the gaming module configured to receive the patient compliance data from a hub different from the device and that generates the patient compliance data in response to receiving indication of ingestion of an ingestible event marker by the patient as part of the therapeutic regimen.

7. The apparatus of claim 1, wherein the gaming module is within a mobile device.

8. The apparatus of claim 1, wherein the modified game data includes data to modify at least one of an appearance of a gaming avatar of the patient, a behavior of the gaming avatar, motion of the gaming avatar, a score associated with the patient, an indication of progress of the patient, or an indication of progress of the gaming avatar.

9. The apparatus of claim 1, wherein the modified game data includes data associated with a measured progress associated with a game goal.

10. An apparatus, comprising:
a hub implemented in a memory or a processing device, the hub configured to receive, from a receiver associated with a patient, an indication associated with ingestion of an ingestible as part of a therapeutic regimen, the hub configured to generate patient compliance data associated with compliance of a patient with a therapeutic regimen based at least in part on the indication, the hub configured to transmit the patient compliance data to a gaming module such that the gaming module modifies a gaming experience of the patient based on the patient compliance data.

11. The apparatus of claim 10, the wherein the indication is associated with ingestion of an ingestible event marker by the patient as part of the therapeutic regimen.

12. The apparatus of claim 10, wherein the hub is configured to receive, from the receiver associated with the patient, the indication in response to the receiver generating the indication in response to detecting ingestion of an ingestible event marker by the patient as part of the therapeutic regimen.

13. The apparatus of claim 10, wherein the hub is configured to transmit physiologic data to the gaming module such that the gaming module modifies the gaming experience of the patient based on the patient compliance data and the physiologic data.

14. The apparatus of claim 10, wherein the hub is configured to transmit non-physiologic data to the gaming module such that the gaming module modifies the gaming experience of the patient based on the patient compliance data and the non-physiologic data.

15. The apparatus of claim 10, wherein the hub is configured to transmit the patient compliance data to the gaming module such that the gaming module modifies a measured progress associated with a game goal.

16. A method, comprising:
receiving, at a gaming module implemented in at least one of a memory or a processing device, patient compliance data associated with compliance of a patient with a therapeutic regimen;
modifying an aspect of an electronic game of the patient based on the patient compliance data to produce modified game data; and
sending a signal associated with the modified game data such that the modified game data is presented to the patient.

17. The method of claim 16, wherein the patient compliance data includes an indication of ingestion of an ingestible event marker by the patient as part of the therapeutic regimen.

18. The method of claim 16, wherein the receiving includes receiving the patient compliance data from a hub that generates the patient compliance data and is implemented in a device different from the gaming module.

19. The method of claim 16, wherein the receiving includes receiving the patient compliance data from a hub that is (1) implemented in a device different from the gaming module and (2) that generates the patient compliance data in response to receiving an indication of ingestion of an ingestible event marker by the patient as part of the therapeutic regimen.

20. The method of claim 16, wherein the modified game data includes data to modify at least one of an appearance of a gaming avatar of the patient, a behavior of the gaming avatar, motion of the gaming avatar, a score associated with the patient, an indication of progress of the patient, or an indication of progress of the gaming avatar.

21. The method of claim 16, wherein the modified game data includes data associated with a measured progress associated with a game goal.
